(19) United States
(12) Patent Application Publication
(10) Pub. No.: US 2012/0245492 A1
(43) Pub. Date: Sep. 27, 2012

(54) REHABILITATION DEVICE FOR PEOPLE WITH PARALYSIS AND OPERATION METHOD THEREOF

(75) Inventor: Yang-Soo Lee, Daegu (KR)

(73) Assignee: Industry-Academic Cooperation Foundation, Kyungpook National University, Daegu (KR)

(21) Appl. No.: 13/354,301

(22) Filed: Jan. 19, 2012

(30) Foreign Application Priority Data
Mar. 22, 2011 (KR) 10-2011-0025442

(51) Int. Cl. A61B 5/103

(52) U.S. Cl. 600/595

(57) ABSTRACT

A rehabilitation device comprises a plurality of sensors configured to detect motion of at least one targeted area of a patient, a display unit to display an image, and a controlling unit configured to determine a motion characteristic of the patient based on output signals from the plurality of sensors, and to control a virtual image representing a human body displayed on the display unit based on the determined motion characteristic. The plurality of sensors of the present invention are engaged with the at least one targeted area.

### CROSS-REFERENCE TO RELATED APPLICATIONS

This application claims priority to and the benefit of Korean Patent Application No. 10-2011-0025442 filed in the Korean Intellectual Property Office on Mar. 22, 2011, the entire contents of which are incorporated herein by reference.

### TECHNICAL FIELD

The present invention relates to a rehabilitation device for a patient with paralysis using a virtual reality or an augmented reality, and an operation method thereof.

### BACKGROUND ART

To treat patients with motor impairment due to aging, disease, industrial accidents, car accidents, and the like, various rehabilitation treatments have been implemented. Recently, all of the rehabilitation treatments are focusing treatment by practicing a task-oriented goal. For example, to treat a patient with a paralyzed hand, a treatment that enables the patient to repeatedly perform a task such as grabbing an object with the paralyzed hand, moving the object, and the like, is implemented. To treat a patient with a paralyzed leg, a treatment that enables the patient to repeatedly perform a functional task such as a stepping up a foothold and the like is implemented.

However, patients with serious paralysis cannot perform such functional task with a paralyzed arm or leg. Even patients with paralysis capable of performing the functional task may lose interest in treatment due to repetition of the predetermined functional tasks.

### SUMMARY OF THE INVENTION

The present invention has been made in an effort to provide a rehabilitation device that helps a rehabilitation treatment of a patient with paralysis by measuring, using a sensor, a minute motion of a patient with paralysis incapable of performing a functional task, and thereby enabling the patient to perform a game and to perform the functional task in a virtual reality using the measurement result, and an operation method thereof.

An exemplary embodiment of the present invention provides a method of operating a rehabilitation device, the method including the step of: detecting a motion of a patient body; determining a motion characteristic of the patient body according to the detected motion; and controlling an image displayed on a monitor according to the determined motion characteristic.

The step of detecting the motion may include the step of detecting an acceleration according to a travel of the patient body and a slope of the patient body with respect to the ground surface.

The step of determining the motion characteristic may include the step of determining bending, straightening, or turning of the patient body.

The step of controlling the image may include the step of controlling the image to perform an operation corresponding to the determined motion characteristic.

The step of controlling the image may be performed by amplifying the determined motion characteristic.

The step of controlling the image may control a virtual image reproducing a human body displayed on the monitor.

The step of controlling the image may be performed when strength of the determined motion characteristic is greater than or equal to a threshold value.

The method may further include the step of gradually increasing the threshold value.

The step of controlling the image may control virtual content augmented on a real background image, according to the determined motion characteristic.

The step of controlling the image may control a game image according to a change in a position of a first body portion (finger) and turning of a second body portion (wrist).

Another exemplary embodiment of the present invention provides a rehabilitation device, including: a plurality of sensors configured to detect motion of at least one targeted area of a patient, the plurality of sensors being engaged with said at least one targeted area; a display unit to display an image; and a controlling unit configured to determine a motion characteristic of the patient based on output signals from the plurality of sensors, and to control a virtual image representing a human body displayed on the display unit based on the determined motion characteristic.

The controlling unit may amplify the determined motion characteristic and may control the virtual image representing the human body according to the amplified motion characteristic.

The controlling unit may be configured to display, on the display unit, a video game that is controlled according to the output signals of the plurality of sensors.

The controlling unit may be configured to evaluate a motion of the patient based on the output signals of the plurality of sensors.

The controlling unit may control virtual content augmented on a real background image, using the human body reproducing image, or may control a game image according to a change in a position of a first body portion and turning of a second body portion.

At least one sensor selected from among the plurality of sensors may be mounted to an elastic member.

Yet another exemplary embodiment of the present invention provides an method of operating a rehabilitation device, the method including the steps of: detecting a motion of a human body; calculating a time period while the motion is terminated after the motion starts, or a speed of the motion through the detection; and controlling an image displayed on a monitor based on the calculated values.

Still another exemplary embodiment of the present invention provides a rehabilitation device, including: a sensor configured to detect a motion of a human body; a display unit to display an image; and a controlling unit to calculate a time period while the motion is terminated after the motion starts, or a speed of the motion through the detection, and to control the image based on the calculated values.

The sensor may be mounted to an elastic member.

Still another exemplary embodiment of the present invention provides a method of operating a rehabilitation device, the method including the steps of: obtaining a depth image about a motion of a patient body; determining a motion characteristic according to the motion by analyzing the depth image; and controlling a virtual image representing a human body displayed on a monitor according to the determined motion characteristic.

According to exemplary embodiments of the present invention, a motion of a body or a joint is detected and thereby is displayed as an image. There are provided a rehabilitation device that provides improved satisfaction since a patient can view a meaningful task performed in a virtual reality by a motion of the patient, and an operation method thereof. Also, the patient needs to perform a game using a motion of a paralyzed body and thus, further moves the paralyzed body.

According to the exemplary embodiments of the present invention, a rehabilitation treatment is performed using a virtual reality. Therefore, there are provided a rehabilitation device that provides improved safety stability, and an operation method thereof.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 illustrates a rehabilitation device according to an exemplary embodiment of the present invention.

FIG. 2 is a flowchart illustrating a first example of an operation method of the rehabilitation device of FIG. 1.

FIG. 3 illustrates an example of sensors attached to a back portion of a user.

FIGS. 4 and 5 illustrate positions of sensors when a user bends and straightens an upper body.

FIG. 6 illustrates positions of sensors when a user turns an upper body counterclockwise.

FIG. 7 illustrates positions of sensors when a user turns an upper body clockwise.

FIG. 8 illustrates an example of sensors attached to a finger of a user.

FIG. 9 illustrates positions of sensors when a user cups fingers.

FIG. 10 illustrates an example of a sensor attached to a wrist of a user.

FIG. 11 illustrates a position of a sensor when a user turns a wrist.

FIG. 12 illustrates an example of sensors attached to a foot of a user.

FIG. 13 illustrates positions of sensors when a user bends and extends an ankle.

FIG. 14 illustrates positions of sensors when a user turns an ankle.

FIG. 15 illustrates an example of sensors attached to a chest of a user.

FIG. 16 illustrates positions of sensors when a user bends an upper body.

FIG. 17 illustrates an example of an image displayed on a display unit.

FIG. 18 illustrates an example in which an image performing a game is displayed on a display unit.

FIG. 19 illustrates a configuration example of a rehabilitation device in an augmented reality environment.

FIG. 20 illustrates an example of a sensor attached to laryngeal prominence of a user.

FIG. 21 illustrates positions of sensor when Laryngeal prominence moves.

FIG. 22 is a flowchart illustrating a second example of an operation method of the rehabilitation device of FIG. 1.

FIG. 23 is a flowchart illustrating a third example of an operation method of the rehabilitation device of FIG. 1.

FIG. 24 is a conceptual diagram illustrating a rehabilitation device according to still another exemplary embodiment of the present invention.

FIGS. 25A and 25B are reference views to describe a motion characteristic determination using a depth image obtained by a capture device of FIG. 24.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter, exemplary embodiments of the present invention will be described with reference to the accompanying drawings to describe in detail the present invention such that those skilled in the art may readily work the technical spirits of the present invention.

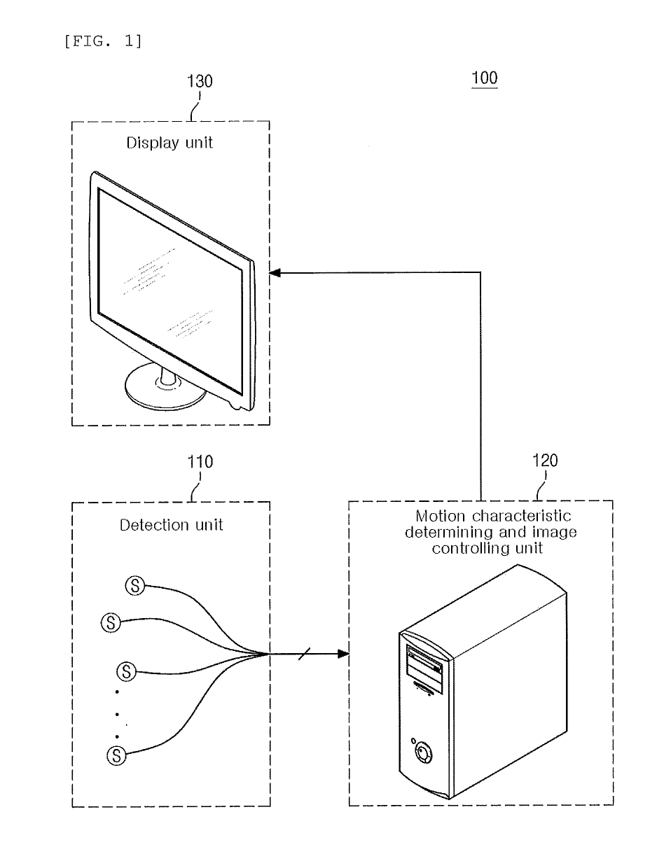

FIG. 1 illustrates a rehabilitation device 100 according to an exemplary embodiment of the present invention. Referring to FIG. 1, the rehabilitation device 100 includes a detection unit 110, a motion characteristic determining and image controlling unit 120, and a display unit 130.

The detection unit 110 includes a plurality of sensors S. The plurality of sensors S detects a motion of a contacted target. For example, the plurality of sensors S may be accelerometers and tilting sensors to detect the motion of the contacted target. The detection result of the plurality of sensors S is transferred to the motion characteristic determining and image controlling unit 120.

The motion characteristic determining and image controlling unit 120 receives the detection result from the detection unit 110. Based on the received detection result, the motion characteristic determining and image controlling unit 120 determines a motion characteristic of the target contacting with the sensors S. The motion characteristic determining and image controlling unit 120 may determine how the target contacting with the sensors S moves. The motion characteristic determining and image controlling unit 120 may determine a motion characteristic such as a movement direction, a turning angle, and the like, of the target contacting with the sensors S.

The motion characteristic determining and image controlling unit 120 controls an image displayed on the display unit 130, according to the determined motion characteristic. The motion characteristic determining and image controlling unit 120 may control the image, which is displayed on the display unit 130, to move according to the determined motion characteristic.

The display unit 130 displays the image according to a control of the motion characteristic determining and image controlling unit 120.

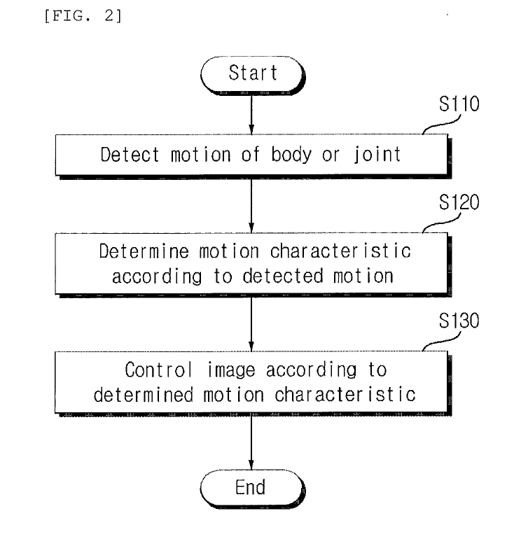

FIG. 2 is a flowchart illustrating a first example of an operation method of the rehabilitation device 100 of FIG. 1. Referring to FIG. 2, in step S110, a motion of a body or a joint is detected. The sensors S of the detection unit 110 may contact with a body of a user (for example, a bone portion beneath a skin). The sensors S may detect the motion of the user. Accelerometers may detect acceleration of the body when the body of the user moves. Camera may detect motion of the body when the body of the user moves. Tilting sensors may detect a slope of the body of the user with respect to the ground surface. The detection result is transferred to the motion characteristic determining and image controlling unit 120.

In step S120, a motion characteristic is determined according to the detected motion. The motion characteristic determining and image controlling unit 120 may determine which joint of the body the user moves, a direction in which the user moves the joint, and a speed at which the user moves the joint.

In step S130, an image is controlled according to the determined motion characteristic. The motion characteristic determining and image controlling unit 120 controls the display unit 130 such that an image of reproducing a human body of the user is displayed. When the motion characteristic is determined based on the detection result of the detection unit 110, the motion characteristic determining and image controlling unit 120 controls the human body reproducing image to move according to the determined motion characteristic. For example, the motion characteristic determining and image controlling unit 120 may control the display unit 130 such that the human body reproducing image displayed on the display unit 130 moves according to the determined motion characteristic.

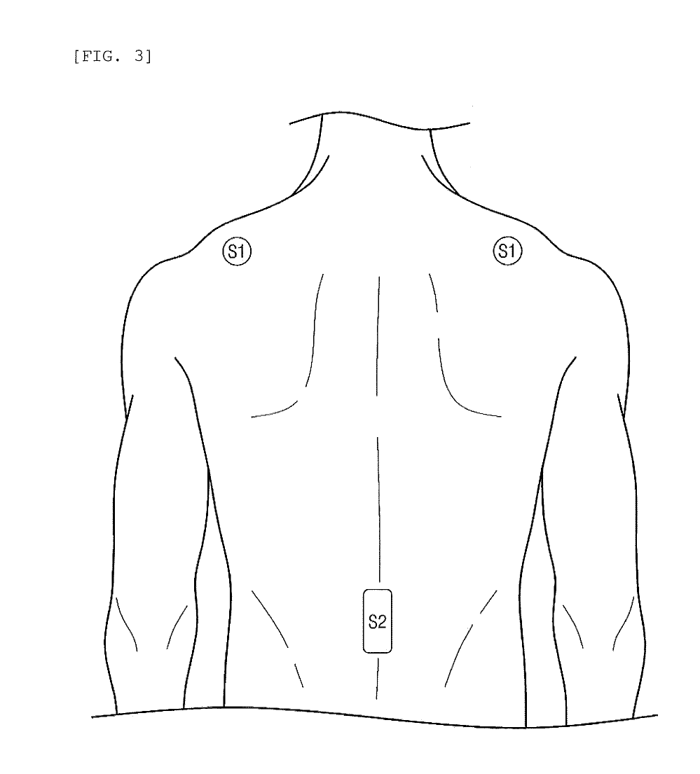

FIG. 3 illustrates an example of sensors attached to a back portion of a user. For example, first sensors S1 may be attached to a shoulder portion, and a second sensor S2 may be attached to a waist. The first sensors S1 may be accelerometers, and the second sensor S2 may be a tilting sensor.

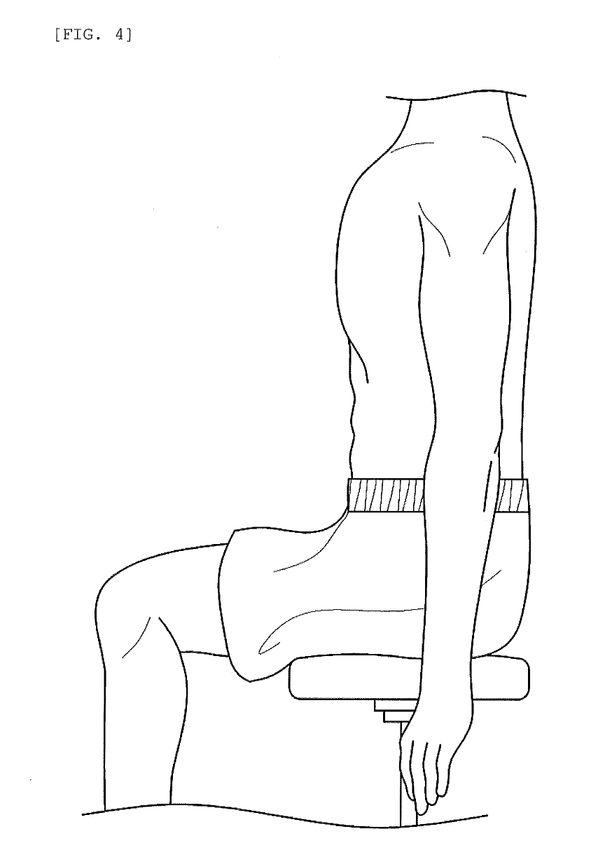

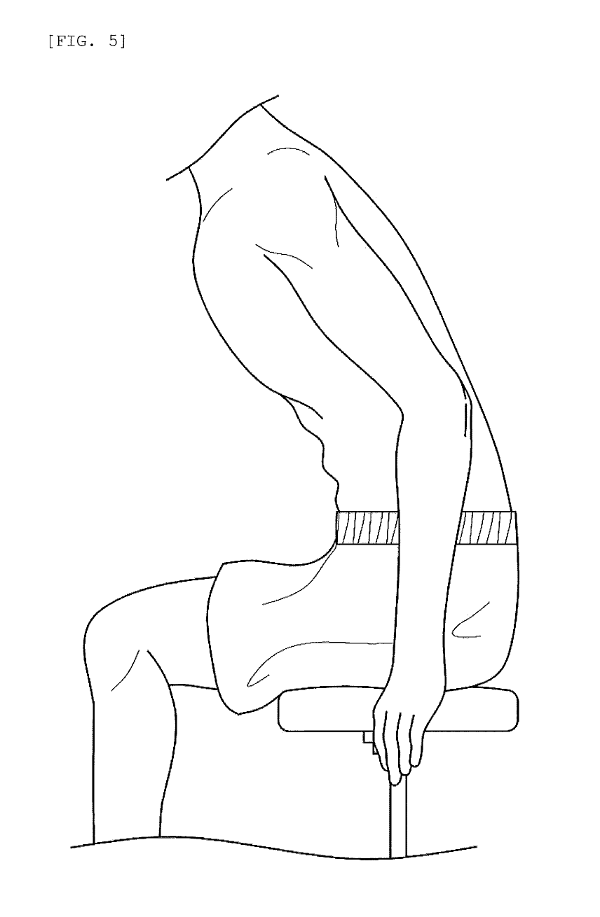

FIGS. 4 and 5 illustrate positions of sensors when a user bends and straightens an upper body. Sensors when the user straightens the upper body are shown in FIG. 4, and sensors when the user bends the upper body are shown in FIG. 5. Referring to FIGS. 4 and 5, a slope of the second sensor S2 of FIG. 4 with respect to the ground surface is different from a slope of the second sensor S2 of FIG. 5 with respect to the ground surface. The second sensor S2 may be a tilting sensor. The motion characteristic determining and image controlling unit 120 may determine whether the user bends or straightens the upper body based on the detecting result of the second sensor S2. The motion characteristic determining and image controlling unit 120 may determine a speed and a degree at which the user bends and straightens the upper body, based on a signal strength of the second sensor S2, a changing speed of a signal thereof, and the like.

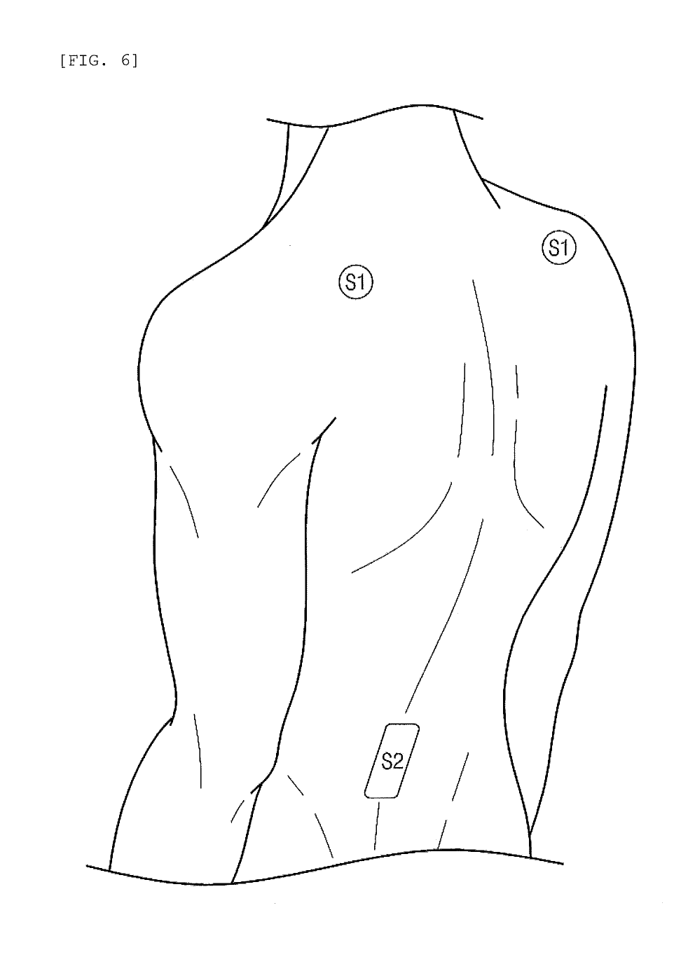

FIG. 6 illustrates positions of sensors when a user turns an upper body counterclockwise. Referring to FIGS. 3 and 6, when the user turns the upper body counterclockwise, the first sensors S1 attached to the shoulder portion of the user turn counterclockwise. The first sensors S1 may be accelerometers. The motion characteristic determining and image controlling unit 120 may determine whether the user turns the upper body counterclockwise based on the detecting result of the first sensors S1. The motion characteristic determining and image controlling unit 120 may determine a speed and a degree at which the user turns the upper body counterclockwise, based on a signal strength of the first sensors S1, a changing speed of a signal thereof, and the like.

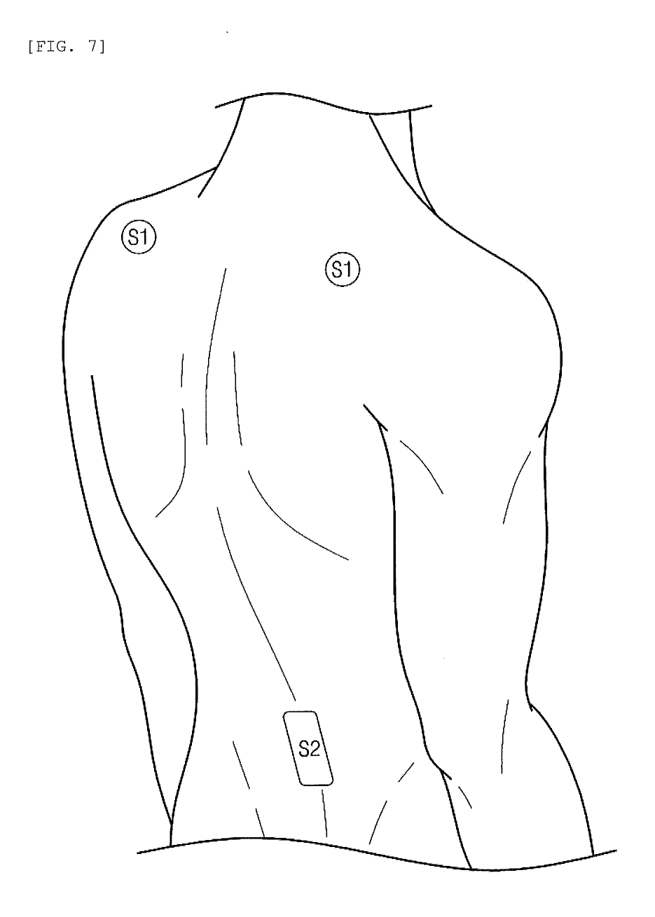

FIG. 7 illustrates positions of sensors when a user turns an upper body clockwise. Referring to FIG. 3 and FIG. 7, when the user turns the upper body clockwise, the first sensors S1 attached to the shoulder portion of the user turn clockwise. The first sensors S1 may be accelerometers. The motion characteristic determining and image controlling unit 120 may determine whether the user turns the upper body clockwise based on the detecting result of the first sensors S1. The motion characteristic determining and image controlling unit 120 may determine a speed and a degree at which the user turns the upper body clockwise, based on a signal strength of the first sensors S1, a changing speed of a signal thereof, and the like.

More effective training may be performed by enabling the user to perform a game and perform a functional task in a virtual reality using signals that are received from a plurality of sensors including the first sensor S1 and the second sensor S2, as well as a single sensor.

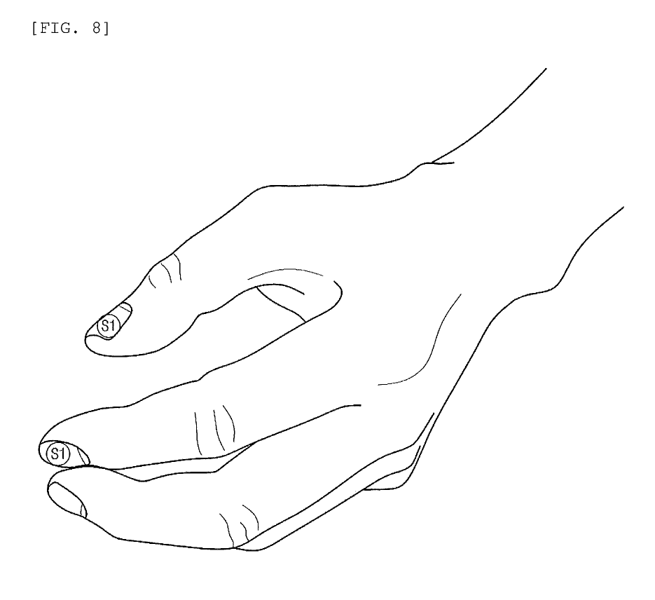

FIG. 8 illustrates an example of sensors attached to a finger of a user. For example, the first sensors S1 are attached to an end portion of a thumb of the user and an end portion of an index finger of the user.

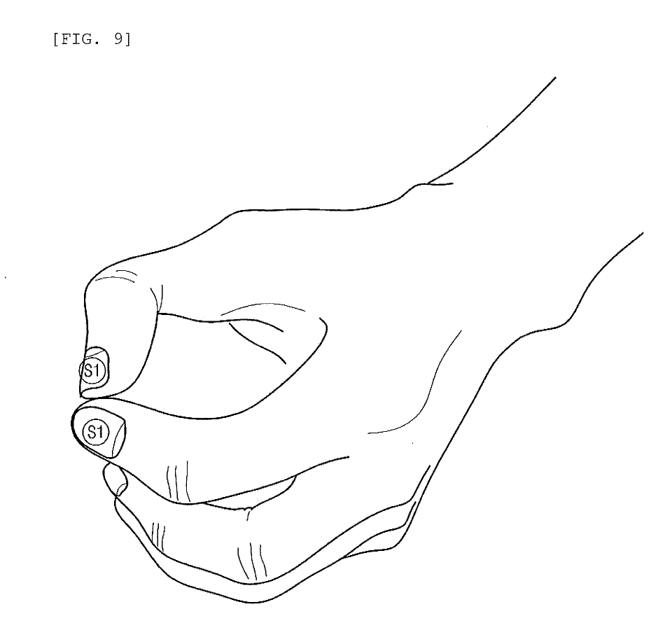

FIG. 9 illustrates positions of sensors when a user flex fingers. Referring to FIGS. 8 and 9, when the user does not cup a thumb and an index finger and when the user cups the thumb and the index finger, positions of the first sensors S1 vary. The first sensors S1 may be accelerometers. The motion characteristic determining and image controlling unit 120 may determine whether the user cups or extend the thumb and the index finger based on the detecting result of the first sensors S1. The motion characteristic determining and image controlling unit 120 may determine a speed and a degree at which the user cups or spreads the thumb and the index finger, based on a signal strength of the first sensors S1, a changing speed of a signal thereof, and the like.

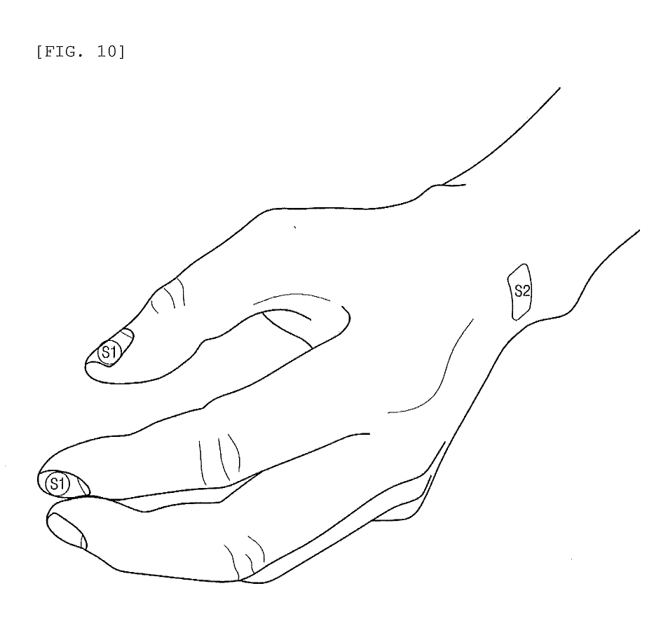

FIG. 10 illustrates an example of a sensor attached to a wrist of a user. For example, the second sensor S2 is attached to a wrist joint portion of the user.

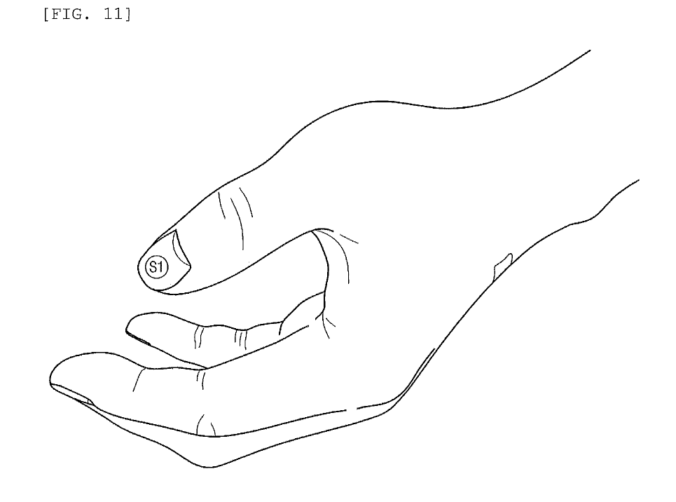

FIG. 11 illustrates a position of a sensor when a user turns a wrist. For example, the position of the sensor when the user turns the wrist counterclockwise is shown in FIG. 11. Referring to FIGS. 10 and 11, before and after the user turns the wrist counterclockwise, slopes of the second sensors S2 with respect to the ground surface vary. The second sensors S2 may be tilting sensors. The motion characteristic determining and image controlling unit 120 may determine whether the user turns the wrist clockwise or counterclockwise based on the detecting result of the second sensors S2. The motion characteristic determining and image controlling unit 120 may determine a speed and a degree at which the user turns the wrist clockwise or counterclockwise, based on a signal strength of the second sensors S1, a changing speed of a signal thereof, and the like.

More effective training may be performed by enabling the user to perform a game and perform a functional task in a virtual reality using signals that are received from the first sensor S1 attached to an end portion of a finger end and the second sensor S2 attached to the wrist.

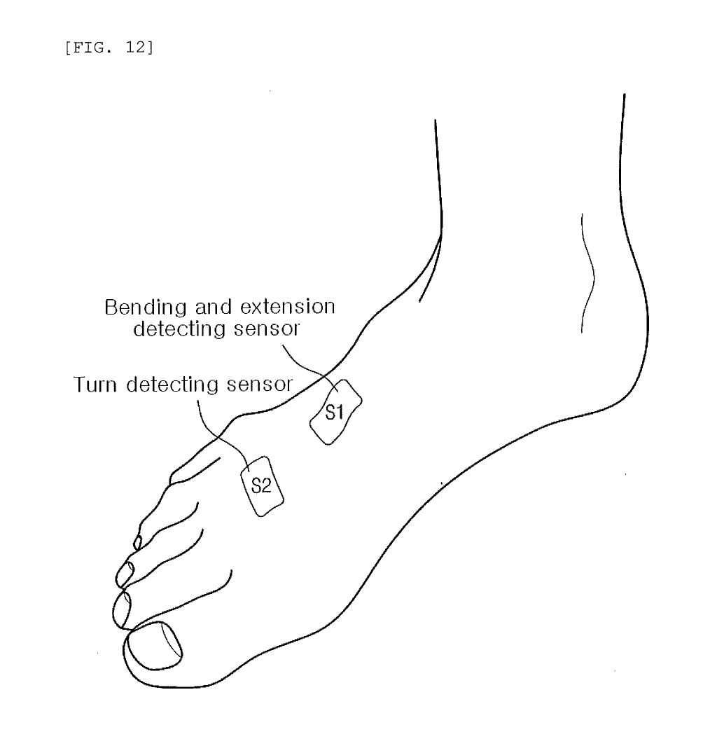

FIG. 12 illustrates an example of sensors attached to a foot of a user. For example, the first sensor S1 and the second sensor S2 are attached to the dorsum of a foot of the user. The first sensor S1 that is disposed in a direction parallel with a bending and extending direction of the foot may be a bending and extension detecting sensor. The second sensor S2 that is disposed in a direction vertical to the bending and extending direction of the foot may constitute a turn detecting sensor.

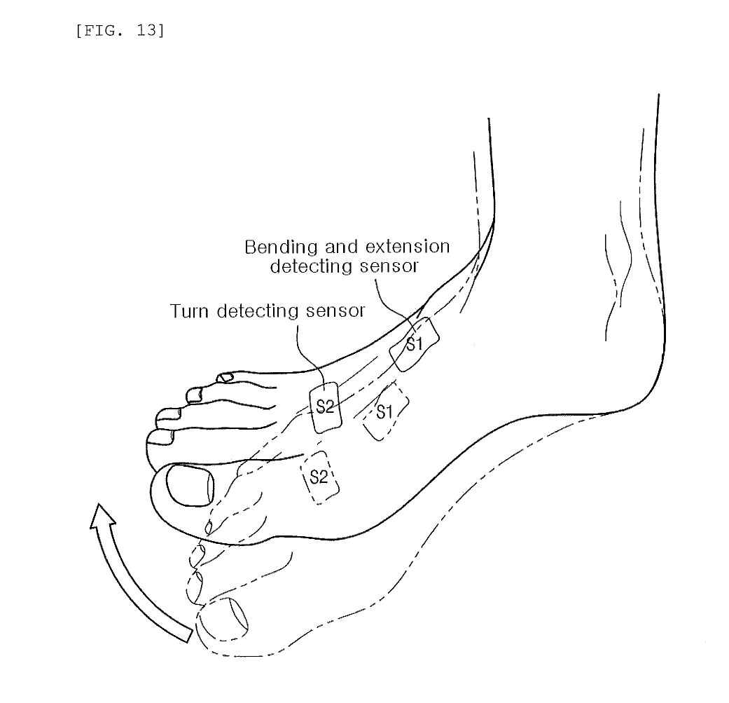

FIG. 13 illustrates positions of sensors when a user bends an ankle. The foot and sensors of FIG. 12 are indicated as a two-dotted chain line and a dashed line, respectively. The foot and sensors of when the user bends the ankle (makes an end of a toe move upward) are indicated as a solid line.

Referring to FIG. 13, when the user bends and extends the ankle, a slope of the first sensor S1, that is, a bending and extension detecting sensor with respect to the ground surface varies. The first sensor S1 that is the bending and extension detecting sensor may be a tilting sensor. The motion characteristic determining and image controlling unit 120 may determine whether the user bending and extension the ankle based on the detecting result of the first sensor S1 that is the bending and extension detecting sensor. The motion characteristic determining and image controlling unit 120 may determine a speed and a degree at which the user bends and extends the ankle, based on a signal strength of the first sensor S1 that is the bending and extension detecting sensor, a changing speed of a signal thereof, and the like.

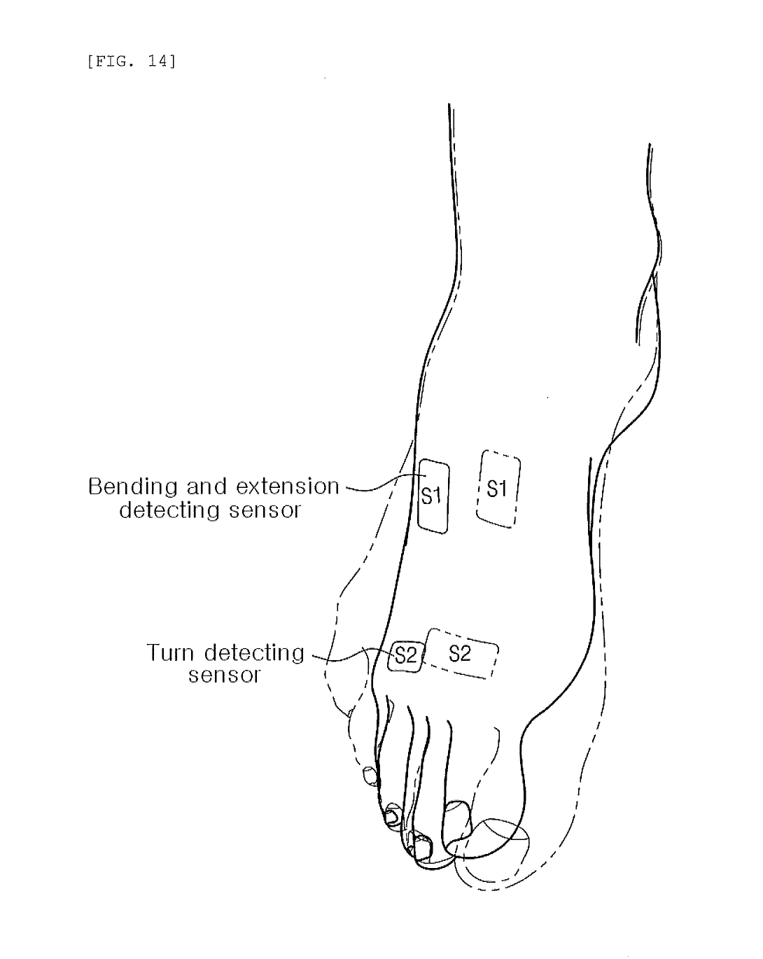

FIG. 14 illustrates positions of sensors when a user turns an ankle. For example, positions of sensors when the user internally turns the ankle (such that a big toe may point upward and a little toe may point downward) are shown in FIG. 14. The foot and the sensors of FIG. 12 are indicated as a two-dotted chain line and a dashed line, respectively. The foot and sensors of when the user internally turns the ankle are indicated as a solid line.

Referring to FIG. 14, before and after the user internally turns the ankle, a slope of the second sensor S2 that is a turn detecting sensor with respect to the ground surface varies. The second sensor S2 that is the turn detecting sensor may be a tilting sensor. The motion characteristic determining and image controlling unit 120 may determine whether the user internally or externally turns the ankle based on the detecting result of the second sensor S2 that is the turn detecting sensor. The motion characteristic determining and image controlling unit 120 may determine a speed and a degree at which the user internally or externally turns the ankle, based on a signal strength of the second sensor S2 that is the turn detecting sensor, a changing speed of the signal thereof, and the like.

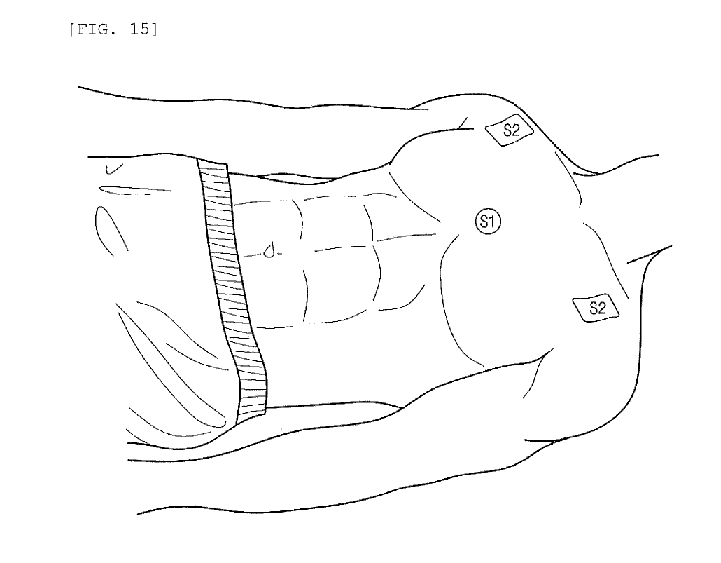

FIG. 15 illustrates an example of sensors attached to a chest of a user. For example, the first sensor S1 may be attached to a center portion of the chest of the user, and the second sensors S2 may be attached to a shoulder portion. The user may lie down on an instrument (for example, a bed and the like) parallel with the ground surface.

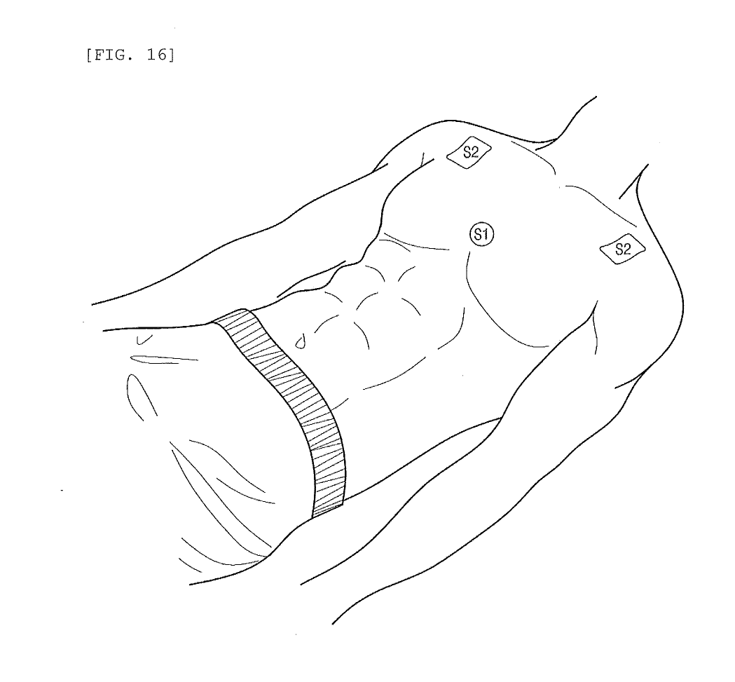

FIG. 16 illustrates positions of sensors when a user bends an upper body. Referring to FIGS. 15 and 16, when the user bends or straightens the upper body, the first sensor S1 moves. The first sensor S1 may be a tilting sensor. The motion characteristic determining and image controlling unit 120 may determine whether the user bends or flex straightens the upper body based on the detecting result of the first sensor S1. The motion characteristic determining and image controlling unit 120 may determine a speed and a degree at which the user bends or straightens the upper body, based on a signal strength of the first sensor S1, a changing speed of a signal thereof, and the like.

As described above with reference to FIGS. 4 and 5, the motion characteristic determining and image controlling unit 120 may determine a speed and a degree at which the user turns the upper body based on the detecting result of the second sensors S2.

As described above, the motion characteristic determining and image controlling unit 120 may determine a direction of a motion of a body or a joint moved by the user, and a magnitude of the motion of the body or the joint, based on the detecting result of sensors that are attached to respective portions of the body or the joint of the user (for example, a patient with paralysis). The motion characteristic determining and image controlling unit 120 may control the image displayed on the display unit 130, based on the determination result.

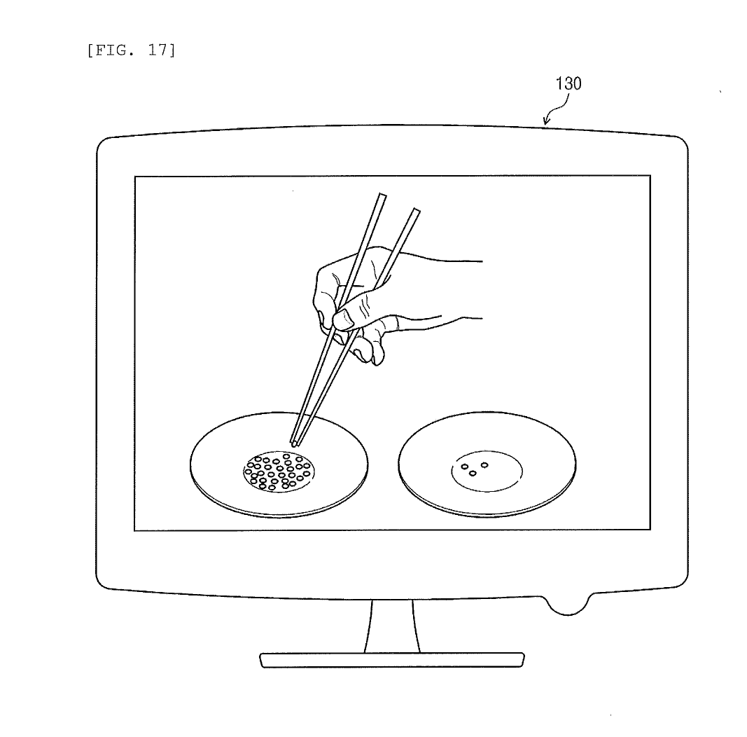

FIG. 17 illustrates an example of an image displayed on the display unit 130. Referring to FIG. 17, two plates and contents placed on the two plates are displayed. For example, the contents placed on two plates may be materials in a form of small particles such as beans. An image of reproducing a body of a user or a portion of the body may be further displayed on the display unit 130. For example, a hand of the user may be displayed on the display unit 130.

The motion characteristic determining and image controlling unit 120 may control a reproducing image displayed on the display unit 130, according to the determination result about the motion of the user. The motion characteristic determining and image controlling unit 120 may amplify the determined motion of the user and thereby display the amplified motion of the user on the display unit 130. For example, even though the user moves only a portion of the finger, the motion characteristic determining and image controlling unit 120 may control the reproducing image, displayed on the display unit 130, to grab or spread chopsticks.

The motion of the user and an operation of the reproducing image may be a functional motion for a rehabilitation training of a patient with paralysis. For example, even the patient with paralysis who cannot pick up chopsticks may perform a task of moving beans placed on one plate to another plate by moving only a portion of a joint motion range of the hand on a screen using the rehabilitation device 100. When moving a paralyzed hand a little by a little, the user may recognize a meaningful motion through the rehabilitation device 100 while viewing a motion of the reproducing image that is displayed on the display unit 130. Accordingly, user motivation with respect to the rehabilitation training may be elevated and the treatment efficiency of the rehabilitation device 100 may be improved. Also, the rehabilitation treatment of the user is performed in a virtual reality that is controlled by the rehabilitation device 100. Accordingly, in the case of the rehabilitation treatment, user safety may be guaranteed.

For example, to increase interest and concentration of the patient with paralysis, the rehabilitation device 100 may display, on the display unit 130, a game that is controlled according to a motion of the patient with paralysis. That is, the rehabilitation device 100 may be a device that performs a game by detecting the motion of the patient.

In the aforementioned exemplary embodiment, an accelerometer or a tilting sensor is attached to a body or a joint of a user. However, the present invention is not limited to the case where a predetermined sensor is attached to a predetermined portion in the body or the joint of the user. The tilting sensor may be attached to a portion that causes a change in a slope when moving the body or the joint of the user. The accelerometer may be attached to a portion that causes a change in a position. At least one of the tilting sensor and the accelerometer may be attached to a portion that causes both the change in the slope and the change in the position.

To detect the motion of the body or the joint of the user, an image sensor (for example, a camera) may be used. The motion of the body or the joint of the user may be detected by photographing an image of the body or the joint of the user and by analyzing the photographed image.

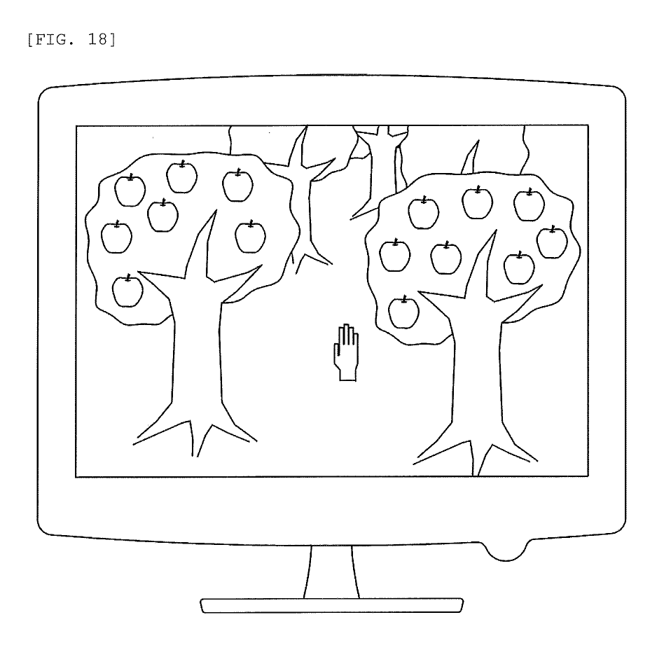

FIG. 18 illustrates an example in which an image of performing a game is displayed on the display unit 130. For example, when the user bends and extends the ankle, a pointer moves upward and downward. When the user internally turns the ankle, the pointer moves to left. When the user externally turns the ankle, the pointer may move to right. A patient having a rehabilitation treatment may practice a motion of picking up an apple as a game by moving the pointer and the like.

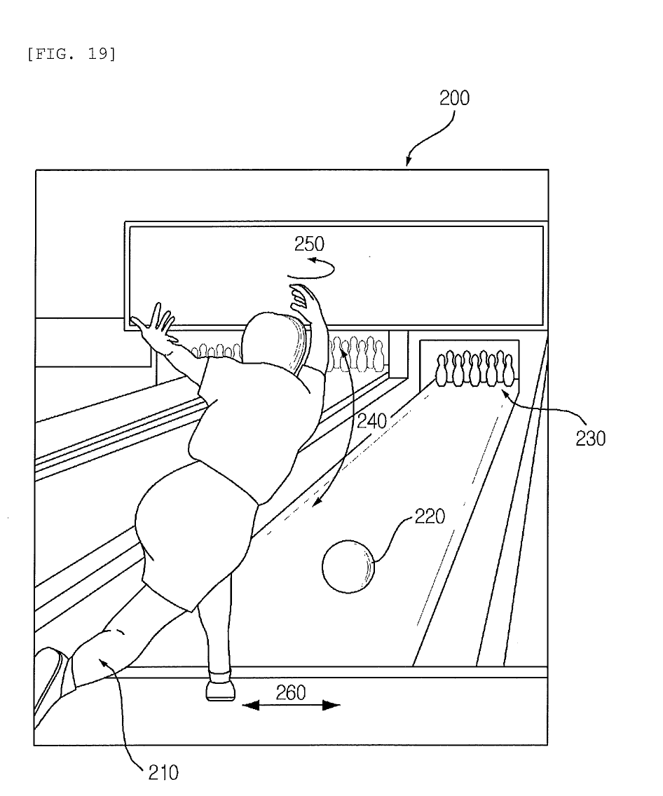

The rehabilitation treatment of the user may be performed in an augmented reality that is controlled by the rehabilitation device 100. FIG. 19 illustrates a configuration example of the rehabilitation device 100 in an augmented reality environment, and illustrates an embodiment in which the user experiences a bowling game according to a motion of the finger and the wrist of the user in the augmented reality environment. Hereinafter, description will be made with reference to FIGS. 8 through 11 and FIG. 19.

The bowling game is a game in which the user rolls a bowling ball and thereby knows down ten pins standing at the end of a lane. In FIG. 19, a user 210 playing the bowling game on an actual image 200 about the bowling alley, a bowling ball 220, pins 230, and the like are augmented as virtual content and thereby are configured as an augmented reality image. To play the bowling game, a speed 240 of the bowl, rotation information 250 of the bowl, a placement position 260 of the bowl, and the like need to be determined. The speed 240 of the bowling ball may be determined by calculating a turning angular speed of a forearm portion using a sensor attached to the forearm portion. The forearm portion indicates a portion from an elbow to a wrist in a human body. The s placement position 240 of the bowling ball may be determined using an accelerometer attached to the finger. For example, as shown in FIGS. 8 and 9, when each of the first sensors S1 that are accelerometers are attached to the end portion of the thumb and the end portion of the index finger of the user, it is possible to measure a change in positions of the first sensors S when extend the thumb and the index finger using the first sensors S, and to determine the placement position 240 of the bowling ball based on the measurement result. The rotation information 250 of the bowling ball includes a rotation speed of the bowl. The turning speed of the bowling ball may be determined based on a slope at the moment when the bowling ball is placed on the floor surface using a tiling sensor that is attached to the hand or the wrist. Here, when the slope is vertical, the rotation speed increases. The placement position 260 of the bowling ball may be determined by detecting the moment when the finger is extended while grabbing the bowling ball using a flexion of the finger.

As described above, when the rehabilitation device 100 is configured in the augmented reality environment, the user may have a feeling as if the user acts on the actual field and thus, it is possible to provide psychological stability to the user during the rehabilitation treatment. In addition, when a game in which the user may play with other people such as the bowling game is configured in the augmented reality environment, it is possible to reduce a sense of alienation that the user feels. To determine the rotation speed of ball, an elaborate motion of the wrist is required and thus, it is possible to achieve the effect of improving the rehabilitation treatment through by playing the bowling game.

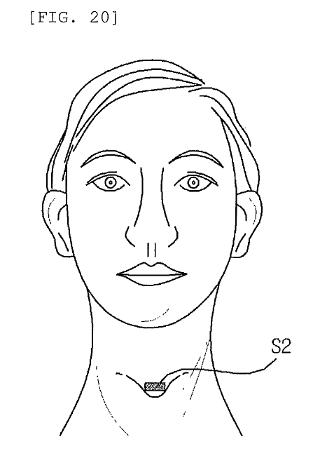

Meanwhile, according to the present exemplary embodiment, it is possible to perform the rehabilitation treatment through a swallowing training. FIG. 20 illustrates an example of a sensor attached to Laryngeal prominence of a user. For example, the second sensor S2 is attached on Laryngeal prominence of the user. The second sensor S2 may measure latency time to laryngeal movement is started, a time used until the motion of Laryngeal prominence is terminated after the motion of Laryngeal prominence starts or a motion speed of Laryngeal prominence through an accelerometer. The second sensor S2 is formed of an elastic material to be attached on the Laryngeal prominence. For example, the second sensor S2 may be formed by mounting a sensor to an elastic member such as medifoam. The second sensor S2 may be formed to cover the overall Laryngeal prominence. The second sensor S2 may be formed in a rectangular form or a quadrate form. However, the present invention is not limited thereto and thus, the second sensor S2 may be formed in a disposable band form.

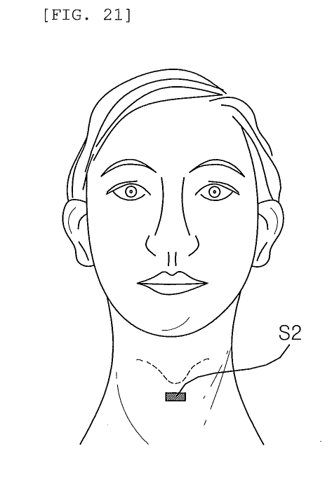

FIG. 21 illustrates positions of sensor when Laryngeal prominence moves. The Laryngeal prominence and the second sensor S2 of FIG. 21 are indicated as a two-dotted chain line and a dashed line, respectively. The Laryngeal prominence and the second sensor S2 of when the user swallows food or saliva are indicated as a solid line.

Referring to FIG. 21, when the Laryngeal prominence moves upward, it is possible to obtain information about a motion by stimulating the second sensor S2 attached to the skin. The motion characteristic determining and image controlling unit 120 may determine whether the user normally performs a swallowing function based on the detecting result of the second sensor S2. Here, the motion characteristic determining and image controlling unit 120 may determine whether the user normally performs the swallowing function based on a time used until an actual motion starts after a signal of inducing a motion of the Laryngeal prominence is provided, a motion speed of Laryngeal prominence, a motion magnitude of Laryngeal prominence, a time used until the motion of Laryngeal prominence is terminated after the motion of Laryngeal prominence starts, and the like. That is, the motion characteristic determining and image controlling unit 120 may determine a speed and a magnitude at which the user performs the swallowing functional based on a signal strength of the second sensor S2, a changing speed of a signal thereof, and the like. When the user swallows food or saliva, the Laryngeal prominence moves upward. Here, the motion of Laryngeal prominence stimulates the second sensor S2, and a result value thereof is displayed in a waveform on the display unit 130. In the case of a person who normally performs the swallowing function, the motion of Laryngeal prominence appears to be fast and great. On the contrary, in the case of a patient with a degraded swallowing function, the motion of Laryngeal prominence and pharynx appears to be slow and weak. Therefore, when the patient views the waveform appearing on the display unit 130 and tries to generate the fast and great waveform intentionally, the effective treatment may be achieved.

The aforementioned swallowing training may be configured as a game. For example, an icon appears to hold a butterfly net in a lower portion below a middle portion of a screen and thereby enable the icon to fly a dragonfly from left to right at various heights. When the user starts moving the Laryngeal prominence after viewing the dragonfly, the butterfly net rises up in the air and the height thereof is proportional to a magnitude of the motion of Laryngeal prominence. When the user moves the Laryngeal prominence fast (response time) and greatly, the user may catch all of the dragonflies.

Meanwhile, the second sensor S2 attached on the Laryngeal prominence may be used for the rehabilitation training of the patient together with the first sensor S1 attached to another body portion.

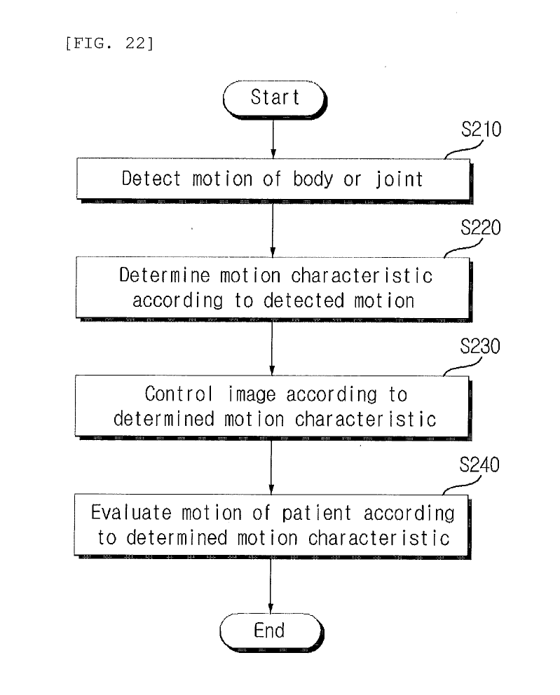

FIG. 22 is a flowchart illustrating a second example of an operation method of the rehabilitation device 100 of FIG. 1. Referring to FIGS. 1 and 22, in step S210, a motion of a body or a joint is detected. In step S220, a motion characteristic is determined according to the detected motion. In step S230, an image is controlled according to the determined motion characteristic. Steps S210 through S230 may be performed using the same method as steps S110 through S130 described above with reference to FIG. 2.

In step S240, a motion of the patient is evaluated according to the determined motion characteristic. For example, the motion of the patient may be evaluated based on a magnitude of a joint motion of a patient with paralysis that is used for a predetermined operation. The evaluation result of the rehabilitation device 100 may be variously used for the rehabilitation treatment of the patient.

For example, to increase interest and concentration of the patient with paralysis, the rehabilitation device 100 may display, on the display unit 130, a game that is controlled according to the motion of the patient with paralysis. That is, the rehabilitation device 100 may be a device that performs a game by detecting a motion of the patient and thereby evaluates a motion of the patient.

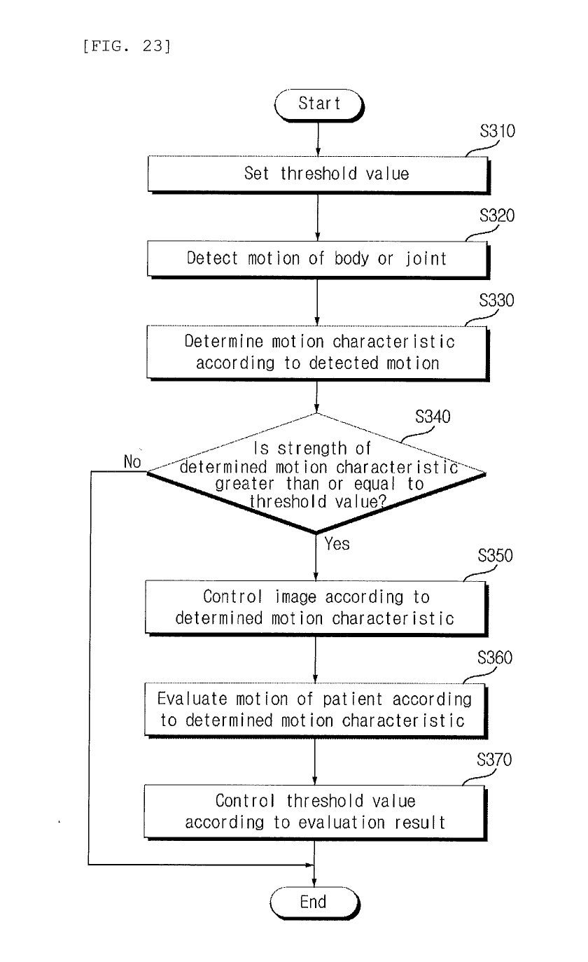

FIG. 23 is a flowchart illustrating a third example of an operation method of the rehabilitation device 100 of FIG. 1. Referring to FIGS. 1 and 23, in step S310, a threshold value is set. In step S320, a motion of a body or a joint is detected. In step S330, a motion characteristic is determined according to the detected motion. Steps S320 and S330 may be performed using the same method as steps S110 and S120 described above with reference to FIG. 2.

In step S340, whether strength of the determined motion characteristic is greater than or equal to the threshold value is determined. When the strength of the determined motion characteristic is less than the threshold value, the motion of the body or the joint of the patient may be ignored. That is, when the strength of force at which the patient moves the body or the joint is less than the threshold value, the rehabilitation device 100 may control a reproducing image, displayed on the display unit 130, not to move.

When the strength of the determined motion characteristic is greater than or equal to the threshold value, the image is controlled according to the determined motion characteristic in step S350. Step S350 may be performed using the same method as step S130 described above with reference to FIG. 2.

In step S360, a motion of the patient is evaluated according to the determined motion characteristic. Step S360 may be performed using the same method as step S240 described above with reference to FIG. 22.

In step S370, the threshold value is controlled according to the evaluation result. When the patient with paralysis moves the body or the joint sufficiently greatly, the threshold value may increase.

For example, rehabilitation steps may be classified according to the strength of force at which the patient with paralysis moves the body or the joint. In each rehabilitation step, a threshold value and a promotion value corresponding to the strength of force at which the patient with paralysis moves the body or the joint may be set. As described above in step S340, the threshold value may be a standard used when the rehabilitation device 100 determines whether to move the reproducing image displayed on the display unit 130. The promotion value may be a standard used when the rehabilitation device 100 determines whether to adjust the threshold value by adjusting the rehabilitation step.

When the magnitude of the motion of the body or the joint of the patient with paralysis reaches the promotion value, the rehabilitation device 100 may promote the rehabilitation step of the patient with paralysis. The patient with paralysis may experience the rehabilitation training with the improved strength by progressing the rehabilitation training according to the raised threshold value. That is, the rehabilitation device 100 may adjust the strength of the rehabilitation training according to a rehabilitation level of the patient with paralysis.

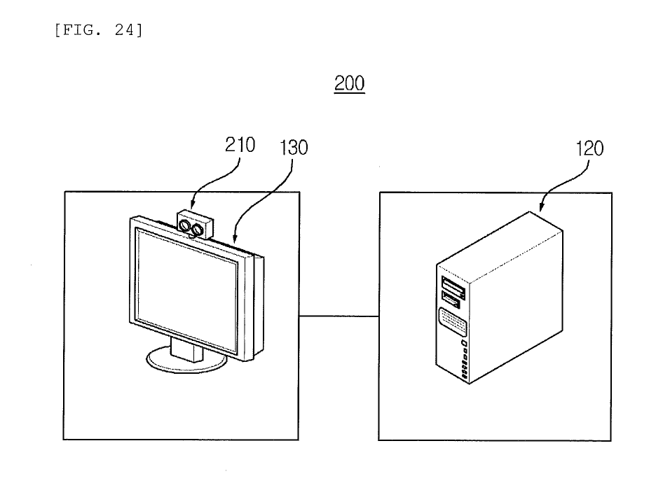

An example in which the detection unit 110 is configured as an accelerometer or a tilting sensor is described above. Here, a depth sensor may also perform a function of the detection unit 110. Hereinafter, the above exemplary embodiment will be described. FIG. 24 illustrates a rehabilitation device 200 according to still another exemplary embodiment of the present invention.

The rehabilitation device 200 according to still another exemplary embodiment of the present invention includes a capture device 210, a motion characteristic determining and image controlling unit 120, and a display unit 130.

Using the capture device 210, the rehabilitation device 200 according to the present exemplary embodiment may determine whether the user bends an upper body, whether the user turns the upper body clockwise or counterclockwise, whether the user flex fingers, whether the user turns a wrist, whether the user bends and extends an ankle, whether the user turns the ankle, whether the user moves Laryngeal prominence, and the like. That is, the capture device 210 of the rehabilitation device 200 according to the present exemplary embodiment may be configured as a function of any one sensor between an accelerometer and a tilting sensor, or may be configured by integrating functions of the accelerometer and the tilting sensor.

The capture device 210 is used to visually monitor the whole body or a predetermined portion (for example, an upper body, a wrist, a finger, an ankle, and the like) of the patient. The capture device 210 is configured to obtain depth images about a body portion of the patient. The capture device 210 may be configured to capture a video having depth information using a predetermined suitable scheme such as a time of flight (TOF), a structured light, a stereo image, and the like. Therefore, the capture device 210 may include a depth camera, a video camera, a stereo camera, and/or other suitable capture devices. Also, to obtain visual stereo data, the capture device 210 may include at least two physically separate cameras that view the patient from different angles. The depth image may include a plurality of observed pixels, and each observed pixel has an observed depth value. The observed depth value includes depth information of the patient that is observed from the capture device 210.

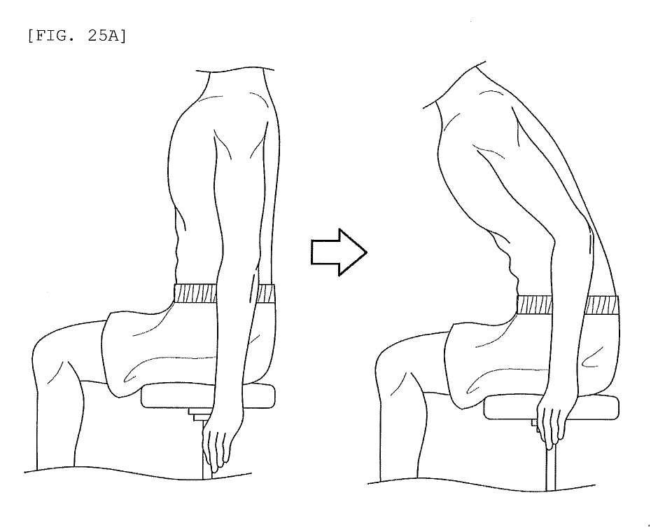

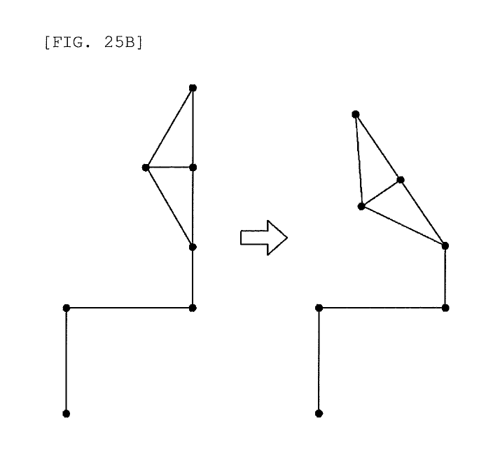

The motion characteristic determining and image controlling unit 120 may be configured to receive the depth image from the capture device 210, and to configure a body portion of the patient as a model. FIGS. 25A and 25B are reference views to describe a motion characteristic determination using a depth image obtained by the capture device 210 of FIG. 24. FIG. 25A shows a case where the user bends and straightens an upper body, and FIG. 25B shows a skeleton model of the patient with respect to the case of FIG. 25A using a depth image. A model including at least two body portions may include at least one joint. Each joint enables at least one body portion to move with respect to at least one another body portion. Also, each body portion of the model may include at least one structural member (that is, “bones”), and joints are positioned at intersecting points of adjacent bones. A portion of bones may correspond to anatomical bones of the patient, and a portion of bones may not correspond to anatomical bones of the patient.

Bones and joints may collectively configure the skeleton model. The skeleton model may be a constituent element of the model. The skeleton model may include at least one skeleton member with respect to each body portion and at least one joint between adjacent skeleton members.

When the patient implements the rehabilitation training, the depth image about a body portion of the patient is obtained by the capture device 210. Here, it is possible to obtain a depth image about the whole body of the patient, including a predetermined body portion of the patient. The motion characteristic determining and image controlling unit 120 forms a skeleton model of the patient using the depth image obtained by the capture device 210. Also, the motion characteristic determining and image controlling unit 120 may detect a change in a motion of the body portion of the patient by analyzing the formed skeleton model of the patient.

As described above, the exemplary embodiments have been described and illustrated in the drawings and the specification. The exemplary embodiments were chosen and described in order to explain certain principles of the invention and their practical application, to thereby enable others skilled in the art to make and utilize various exemplary embodiments of the present invention, as well as various alternatives and modifications thereof. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A method of operating a rehabilitation device, the method comprising the steps of:
detecting a motion of a patient body;
determining a motion characteristic of the patient body according to the detected motion; and
controlling an image displayed on a monitor according to the determined motion characteristic.

2. The method of claim 1, wherein the step of detecting the motion further comprises the step of detecting an acceleration according to a movement of the patient body and a slope of the patient body with respect to the ground surface.

3. The method of claim 1, wherein the step of determining the motion characteristic further comprises the step of determining bending, straightening, or turning of the patient body.

4. The method of claim 1, wherein the step of controlling the image further comprises the step of controlling the image to perform an operation corresponding to the determined motion characteristic.

5. The method of claim 4, wherein the step of controlling the image is performed by amplifying the determined motion characteristic.

6. The method of claim 4, wherein the step of controlling the image controls a virtual image representing a human body displayed on the monitor.

7. The method of claim 1, wherein the step of controlling the image is performed when strength of the determined motion characteristic is greater than or equal to a threshold value.

8. The method of claim 7, further comprising the step of gradually increasing the threshold value.

9. The method of claim 1, wherein the step of controlling the image controls virtual content augmented on a real background image, according to the determined motion characteristic.

10. The method of claim 1, wherein the step of controlling the image controls a game image according to a change in a position of a first body portion and turning of a second body portion.

11. A rehabilitation device, comprising:
a plurality of sensors configured to detect motion of at least one targeted area of a patient, the plurality of sensors being engaged with said at least one targeted area;
a display unit to display an image; and
a controlling unit configured to determine a motion characteristic of the patient based on output signals from the plurality of sensors, and to control a virtual image representing a human body displayed on the display unit based on the determined motion characteristic.

12. The device of claim 11, wherein the controlling unit amplifies the determined motion characteristic and controls the virtual image representing the human body according to the amplified motion characteristic.

13. The device of claim 11, wherein:
the controlling unit is configured to display, on the display unit, a video game that is controlled according to the output signals of the plurality of sensors.

14. The device of claim 13, wherein the controlling unit is configured to evaluate a motion of the patient based on the output signals of the plurality of sensors.

15. The device of claim 11, wherein the controlling unit controls virtual content augmented on a real background image, using the human body reproducing image, or controls a game image according to a change in a position of a first body portion and turning of a second body portion.

16. The device of claim 11, wherein at least one sensor selected from among the plurality of sensors is mounted to an elastic member.

17. A method of operating a rehabilitation device, the method comprising the steps of:
detecting a motion of a human body;
calculating a value relating to a time period while the motion is terminated after the motion starts, or to a speed of the motion through the detection; and
controlling an image displayed on a monitor based on the calculated value.

18. A rehabilitation device, comprising:
a sensor configured to detect a motion of a human body;
a display unit to display an image; and
a controlling unit to calculate a value relating to a time period while the motion is terminated after the motion starts, or to a speed of the motion through the detection, and to control the image based on the calculated value.

19. A method of operating a rehabilitation device, the method comprising the steps of:
obtaining a depth image about a motion of a patient body;
determining a motion characteristic according to the motion by analyzing the depth image; and
controlling a virtual image representing a human body displayed on a monitor according to the determined motion characteristic.

20. A method of operating a rehabilitation device, the method comprising the steps of:
obtaining a visual image about a motion of a patient body using a camera imaging device;
determining a motion characteristic according to the motion by analyzing the visual image; and
controlling a virtual image representing a human body displayed on a monitor according to the determined motion characteristic.
